(19) United States
(12) Patent Application Publication
(10) Pub. No.: US 2004/0029810 A1
(43) Pub. Date: Feb. 12, 2004

(54) Process for preparing distamycin derivatives

(76) Inventors: Italo Beria, Milan (IT); Paolo Cozzi, Milan (IT); Nicola Mongelli, Milan (IT); Marcella Nesi, Saronno (IT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/312,581

(22) PCT Filed: Jul. 12, 2001
(86) PCT No.: PCT/EP01/08031

(30) Foreign Application Priority Data
Jul. 20, 2000 (GB) 0017852.5

(51) Int. Cl. A61K 38/05; C07K 5/06

(52) U.S. Cl. 514/18; 530/331

(57) ABSTRACT

Herewith provided is a process for preparing useful intermediates in the preparation of distamycin derivatives possessing antitumor activity, said derivatives having the formula reported in the specification, by using distamycin A as the starting material.

The present invention relates to a process for preparing distamycin derivatives and, more in particular, to a process for preparing a key intermediate in the preparation of a variety of distamycin derivatives bearing nitrogen-containing ending groups and possessing valuable biological properties as antitumor agents.

Distamycin A, whose formula is reported below

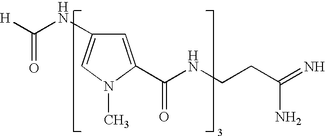

belongs to the family of the pyrroleamidine antibiotics and it is reported to interact reversibly and selectively is with DNA-AT sequences, thus interfering with both replication and transcription. See, for a reference, Nature, 203, 1064 (1964); FEBS Letters, 7 (1970) 90; Prog. Nucleic Acids Res. Mol. Biol., 15, 285 (1975).

Several distamycin derivatives are known in the art as antitumor agents.

As an example, the international patent application WO 98/04524 in the name of the applicant itself, discloses novel distamycin derivatives possessing antitumor activity, wherein the distamycin formyl group is replaced by an acryloyl moiety and the amidino residue is replaced by several nitrogen-containing ending groups among which is guanidino.

These latter compounds, hereinafter shortly referred to as distamycin-guanidines, are also disclosed in the following patent applications WO 97/28123, WO 97/43258, WO 99/50265, WO 99/50266, WO 99/64413 and WO 01/40181 (claiming priority from British patent application No. 9928703.9), all in the name of the applicant itself, and herewith incorporated by reference.

Representative of this class of compounds optionally in the form of pharmaceutically acceptable salts are, for instance:

1) N-(5-{[(5-{[(5-{[(2-{[amino(imino)methyl]amino}ethyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide;

2) N-(5-{[(5-{[(5-{[(2-{[amino(imino)methyl]amino}ethyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-chloroacryloyl)amino]-1-methyl-1H-pyrrole-2-carboxamide;

3) N-(5-{[(5-{[(5-{[(2-{[amino(imino)methyl]amino}ethyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-imidazole-2-carboxamide;

4) N-(5-{[(5-{[(5-{[(2-{[amino(imino)methyl]amino}ethyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide;

5) N-(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)-4-({[4-({4-[bis(2-chloroethyl)amino]benzoyl} amino)-1-methyl-1H-pyrrol-2-yl]carbonyl}amino)-1-methyl-1H-pyrrole-2-carboxamide;

6) N-(5-{[(5-{[(5-{[(2-{[amino(imino)methyl]amino}ethyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-({4-[bis(2-chloroethyl)amino]benzoyl}amino)-1-methyl-1H-pyrazole-5-carboxamide;

7) N-(5-{[(5-{[(2-{[amino(imino)methyl]amino}ethyl)amino]carbonyl}-1-methyl-1H-pyrrol-3-yl)amino)carbonyl}-1-methyl-1H-pyrrol-3-yl)-4-[({4-[(-3-{4-[bis(2-chloroethyl)amino)phenyl}-2-propenoyl)amino]-1-methyl-1H-pyrrol-2-yl}carbonyl)amino]-1-methyl-1H-pyrrole-2-carboxamide.

Several other distamycin derivatives possessing nitrogen-containing ending groups other than guanidino, for instance amino, amidino and amido groups, are widely known in the art as antitumor agents.

All of these compounds, and analogues thereof, are prepared according to a known chemical process comprising, essentially, the condensation reaction between a properly activated carboxylic acid derivative with a poly-pyrroleamido framework bearing the desired nitrogen-containing ending group.

This latter poly-pyrroleamido intermediate, in its turn, is prepared according to a rather troublesome step-by-step procedure which implies, substantially, several acylation reactions of 2-carboxy-4-amino-pyrroles which are obtained through reductions of the corresponding nitro derivatives, in a serial manner.

For a general reference to the above process for preparing distamycin derivatives and poly-pyrroleamido intermediates see, for instance, the aforementioned WO 98/04524 patent application.

In this respect, we have surprisingly found that the said poly-pyrroleamido derivatives can be advantageously prepared through a process which, by starting from distamycin A itself, allows to obtain the desired products in high yields and purity and according to a limited number of steps.

Therefore, it is a first object of the present invention a process for preparing a poly-pyrroleamido derivative of formula (I)

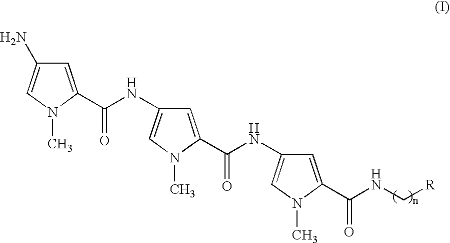

wherein n is an integer from 1 to 4 and R is selected from the group consisting of

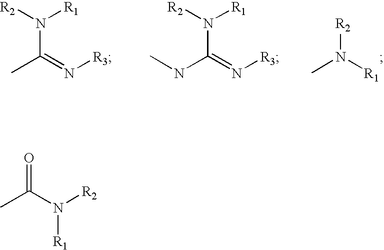

wherein R₁, R₂ and R₃, the same or different, are independently selected from hydrogen, methyl or ethyl;

which process comprises:

a) reacting distamycin A with succinic anhydride in the presence of a base so as to obtain a compound of formula

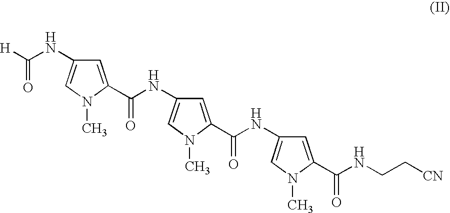

b) reacting the compound of formula (II) with tert-butyl-dicarbonate, in the presence of dimethylaminopyridine (DMAP), so as to obtain a compound of formula

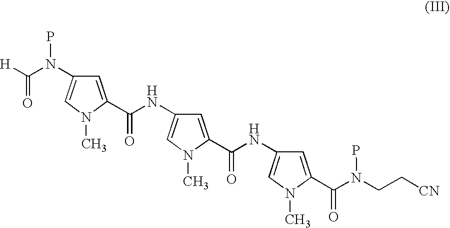

wherein P is tert-butoxycarbonyl;

c) hydrolysing under basic conditions the compound of formula (III) so as to obtain the derivative of formula

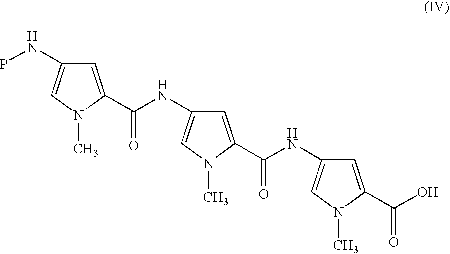

wherein P has the above reported meanings;

d) reacting, in the presence of a suitable coupling agent, the compound of formula (IV) with a compound of formula

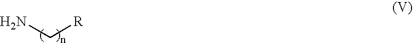

wherein n and R have the above reported meanings; and

e) deprotecting the resultant compound so as to obtain the free amino derivative of formula (I).

The process object of the present invention allows to obtain the compounds of formula (I), as useful intermediates in the synthesis of a variety of distamycin derivatives, under mild conditions and in high yields and purity.

In addition, it enables the preparation of the aforementioned compounds without the need of carrying out several steps and/or isolating intermediate amino derivatives which could lead to undesired by-products.

According to a preferred embodiment of the invention, the present process may be advantageously applied to the preparation of compounds of formula (I) wherein R is a group of formula

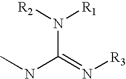

wherein R₁, R₂ and R₃, the same or different, are independently selected from hydrogen, methyl or ethyl. Even more preferably, the above R₁, R₂ and R₃ groups are all hydrogen atoms.

The compound of formula (II), in step a) of the process of the invention, can be obtained by treating commercially available distamycin A with succinic anhydride, preferably as a slight excess, in the presence of a conventional base, for instance sodium or potassium carbonate.

The reaction is carried out in an organic solvent, preferably dimethylformamide (DMF), for a time varying from about 2 to about 48 hours and at a temperature varying from about 20° C. to about 100° C.

In step b), the compound of formula (III) can be obtained by reacting the compound of formula (II) with from about 2 to about 4 equivalents of di-tert-butyldicarbonate, in the presence of from about 2 to about 4 equivalents of dimethylaminopyridine (DMAP), and with a slight excess of an organic base, preferably triethylamine (TEA).

The above reaction is preferably carried out in an organic solvent, for instance DMF, at a temperature varying from about 0° C. to about 30° C. and for a time varying from about is 1 to about 24 hours.

In step c), the hydrolysis of the compound of formula (III) to obtain the compound of formula (IV) is carried out under basic conditions, for instance with an excess of an inorganic base, e.g. sodium or lithium hydroxide, in a mixture of water/organic solvent such as dioxane, acetonitrile or, preferably, tetrahydrofuran (THF).

The reaction temperature may vary from about 0° C. to about 70° C. and for a time varying from about 1 to about 24 hours.

In step d), the reaction between the compound of formula (IV) and the compound of formula (V) can be carried out in an organic solvent, preferably DMF, in the presence of a suitable coupling agent such as, for instance, N,N′-dicyclohexylcarbodiimide (DCC), 1-(3-dimethylaminopropyl)-3-ethylcarbodiimide hydrochloride (EDCI), benzotriazol-1-yloxytris(dimethylamino)phosphonium hexafluorophosphate (BOP) or, preferably, O-(1H-benzotriazol-1-yl)-N,N,N′,N′-tetramethyluronium tetrafluoroborate (TBTU), in the presence of an organic base, e.g. diisopropylethylamine (DIPEA), pyridine or preferably triethylamine (TEA).

The reaction temperature may vary from about −20° C. to about 40° C. and for a time varying from about 5 to about 24 hours.

The removal of the amino protecting tert-butoxycarbonyl group, so as to obtain the corresponding compound of formula (I), in step e), is carried out according to conventional techniques widely known in organic chemistry. As an example, the above deprotection reaction may be carried out under acidic conditions, for instance by using hydrochloric or trifluoroacetic acid, in an organic solvent such as dichloromethane, ethanol or preferably methanol, at a temperature varying from about −20° C. to about 40° C.

From the foregoing, it is clear to the man skilled in the art that by carrying out the deprotection reaction as above indicated, for instance in the presence of hydrochloric acid, the corresponding amino derivative of formula (I) as an acid addition salt with hydrochloric acid, is obtained.

Likewise, for any purpose for which it is desired, the compounds of formula (I), either as such or as acid addition salts, may be conveniently converted into corresponding amino protected derivatives according to conventional techniques. Suitable amino protecting groups known in the art are, for instance, formyl, benzyloxycarbonyl or tert-butoxycarbonyl.

All of the reagents of the above process, object of the present invention, are commercially available or easily preparable according to known methods.

As an example, the compounds of formula (V) wherein R has the above meanings may be prepared, for instance, as reported in J. Amer. Chem. Soc. 81, 1959, 4328; J. Chem. Soc. 1961, 5120-5127; or Chem. Ber. 97, 1964, 704-708. Likewise, the starting material distamycin A can be prepared according to a microbiological process as described, for instance, by Arcamone et al. in Nature 203, 1064 (1964).

As set forth above, the compounds of formula (I) are useful intermediates in the preparation of distamycin derivatives possessing antitumor activity.

It is therefore a further object of the invention a process for preparing distamycin derivatives of formula

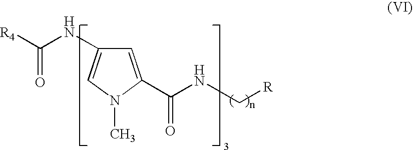

wherein

n is an integer from 1 to 4;

R is selected from the group consisting of

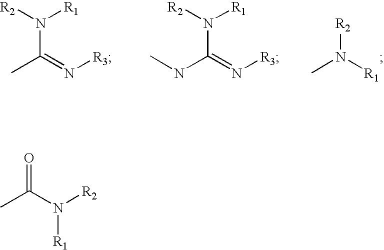

wherein R₁, R₂ and R₃, the same or different, are independently selected from hydrogen, methyl or ethyl;

R₄ is selected from the group consisting of:

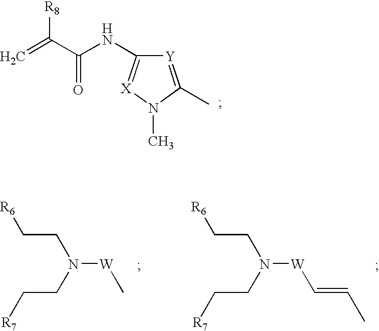

wherein

R₅ and R₆ are chlorine or bromine atoms;

R₇ is hydrogen, chlorine or bromine;

X and Y, the same or different, are selected from nitrogen atoms or CH groups;

W is phenylene or a benzocondensed 5 or 6 membered heterocycle with 1 or 2 heteroatoms selected among N, O or S, both of which being optionally further substituted by lower alkyl groups;

or pharmaceutically acceptable salts thereof;

which process comprises:

a) reacting distamycin A with succinic anhydride in the presence of a base so as to obtain a compound of formula

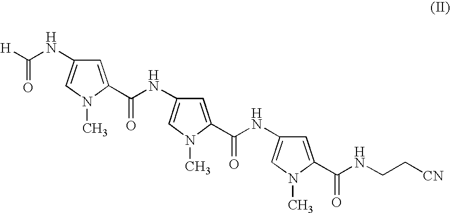

b) reacting the compound of formula (II) with tert-butyl-dicarbonate, in the presence of dimethylaminopyridine (DMAP), so as to obtain a compound of formula

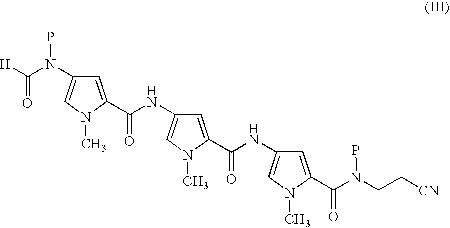

wherein P is tert-butoxycarbonyl;

c) hydrolysing under basic conditions the compound of formula (III) so as to obtain the derivative of formula

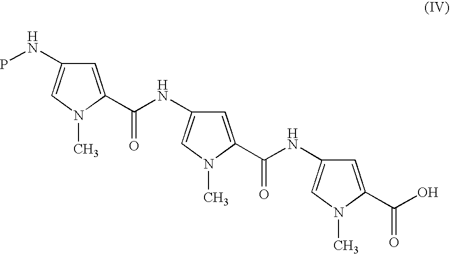

wherein P has the above reported meanings;

b) reacting, in the presence of a suitable coupling agent, the compound of formula (IV) with a compound of formula

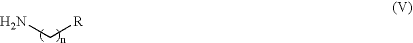

wherein n and R have the above reported meanings; and

e) deprotecting the resultant compound so as to obtain the free amino derivative of formula (I)

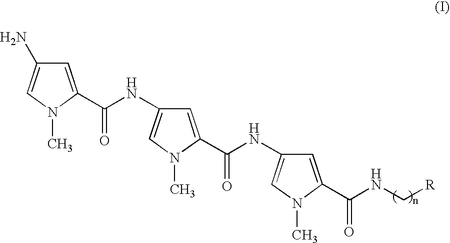

wherein n and R have the above reported meanings; and

f) acylating the compound of formula (I) with a carboxylic acid derivative of formula

R₄—COZ (VII)

wherein R₄ has the above reported meanings and Z is hydroxy or a suitable leaving group, so as to obtain the compounds of formula (VI) and, whenever desired, converting them into pharmaceutically acceptable salts thereof.

The acylation reaction according to step f) between the compound of formula (I) and of formula (VII) is carried out according to conventional techniques.

As an example, the reaction between a compound of formula (I) and a compound of formula (VII) wherein Z is hydroxy, is preferably carried out in an organic solvent, e.g. dimethylsulfoxide, dimethylformamide, ethanol, benzene or pyridine, in the presence of an organic or inorganic base, e.g. triethylamine, diisopropylethylamine, sodium or potassium carbonate or bicarbonate, and in the presence of a condensing agent, e.g. N-ethyl-N′-dicyclohexylcarbodiimide and/or hydroxybenzotriazole hydrate.

The reaction temperature may vary from about −10° C. to about 100° C. and for a time of about 1 hour to about 24 hours.

Analogous operative conditions apply when using the compounds of formula (VII) wherein Z is a halogen atom, preferably bromine or chlorine.

The reaction, in particular, is carried out in an organic solvent such as dimethylformamide, dioxane, pyridine, benzene, tetrahydrofuran, or aqueous admixtures thereof, optionally in the presence of a base.

The reaction is carried out at temperatures varying from about 0° C. to about 100° C. and for a time varying from about 2 to about 48 hours.

The optional conversion of a compound of formula (VI) into a pharmaceutically acceptable salt thereof may be carried out by conventional methods.

Examples of pharmaceutically acceptable salts of the compounds of formula (VI) are the salts with pharmaceutically acceptable inorganic or organic acids such as, for instance, hydrochloric, hydrobromic, sulfuric, nitric, acetic, trifluoroacetic, propionic, succinic, malonic, citric, tartaric, methanesulfonic, p-toluensulfonic and the like.

The intermediate compounds of formula (VII) are known or easily prepared according to known methods, for instance as reported in the aforementioned patent applications. According to this latter aspect of the invention, preferred distamycin derivatives thus preparable are those wherein R represents a group of formula

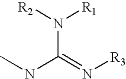

wherein R₁, R₂ and R₃, the same or different, are selected from hydrogen, methyl or ethyl; R₄ is an α-bromo- or α-chloro-acryloyl moiety of formula

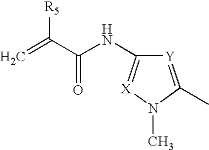

wherein R₅ is a chlorine or bromine atom; X and Y, the same or different, are selected from nitrogen atoms or CH groups.

Particularly preferred, among the distamycin derivatives of formula (I) preparable according to the process object of the invention, is the aforementioned N-(5-{[(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromo acryloyl)amino]-1-methyl-1H-pyrrole-2-carboxamide. According to a preferred embodiment of the invention for its preparation, a proper amount of distamycin A is reacted under basic conditions with a slight excess of succinic anhydride, so as to obtain the corresponding derivative of formula (II), which is then reacted with a proper amount of di-tert-butyl-dicarbonate in the presence of a proper amount of dimethylaminopyridine.

Preferably, in the above reaction, from 2 to 4 equivalents of di-tert-butyldicarbonate and from 2 to 4 equivalents of dimethylaminopyridine are used.

The thus obtained compound of formula (III) is then hydrolysed under basic conditions and subsequently reacted with a proper amount of N-(2-aminoethyl)guanidine of formula (V).

The resultant compound, further deprotected according to conventional techniques, for instance in the presence of hydrochloric acid, yields the corresponding poly-pyrroleamido framework of formula (I) bearing the desired guanidino ending group wherein R₁, R₂ and R₃ are all hydrogen atoms.

The compound of formula (I) is then reacted with a suitable amount, for instance in a molar ratio (I):(VII) comprised from 1:1 to 1:2, of 1-methyl-4-(α-bromoacryloylamido)pyrrole-2-carbonyl chloride of formula (VII), so as to obtain the desired compound.

With the aim of illustrate the present invention without posing any limitation to it, herewith provided are the following examples.

### EXAMPLE 1

Preparation of N-(5-{[(5-{[(2-cyanoethyl)amino]carbonyl}-1-methyl-1H-pyrrol-3-yl)aminolcarbonyl}-1-methyl-1H-pyrrol-3-yl)-4-formyl-1-methyl-1H-pyrrole-2-carboxamide

To a solution of distamycin A (12.0 g) in DMF (240 ml), succinic anhydride (5.8 g) and Na₂CO₃ (8.0 g) were added. The suspension was warmed to 70° C. and stirred for 4 hours. The organic solvent was allowed under vacuum, the residue treated with water (60 ml) and the suspension cooled at 5° C. overnight. The suspension was then filtered under vacuum yielding, after vacuum desiccation at 40° C., the title compound (9,4 g; y=87%) as a hazel powder.

FAB-MS: m/z 465(100, [M+H]⁺)

PMR (DMSO-d₆) δ: 10.05 (s, 1H), 9.95 (s, 2H), 8.25 (t, J=S5.1 Hz, 1H), 8.09 (s, 1H), 7.23 (d, J=1.8 Hz, 1H), 7.21 (d, J=1.8 Hz, 1H), 7.19 (d, J-=1.8 Hz, 1H) 7.02 (d, J=1.8 Hz, 1H), 6.97 (d, J=1.8 Hz, 1H), 6.94 (d, J=1.8 Hz, 1H), 3.82 (s, 3H), 3.81 (s, 3H), 3.79 (s, 3H), 3.42-3.28 (m, 2H), 2.75-2.68 (m, 2H).

### EXAMPLE 2

Preparation of tert-butyl 5-{[(5-{[(5-{[(tert-butoxy carbonyl)(2-cyanoethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl(formyl)carbamate

To a solution of the compound prepared according to example 1 (4.64 g) in dry DMF (20 ml) cooled at 5° C., di-tert-butyldicarbonate (8.73 g), TEA (2.78 ml) and DMAP (2.44 g) were added. The solution was stirred for 5 hours and the organic solvent was then allowed under vacuum. The residue was purified by flash chromatography (dichloromethane/ethyl acetate=6:4) yielding the title compound (2.61 g; y=40%) as a beige powder.

FAB-MS: m/z 665(100, [M+H]⁺); 565(20); 465 (55)

PMR (DMSO-d₆) δ: 9.97 (s, 1H), 9.89. (s, 1H), 9.25(s, 1H), 7.49 (d, J=1.8 Hz, 1H), 7.20 (d, J=1.8 Hz, 1H), 7.06 (d, J=1.8 Hz, 1H), 6.99 (d, J=1.8 Hz, 1H), 6.85 (d, J=1.8 Hz, 1H), 6.65 (d, J=1.8 Hz, 1H), 3.87 (s, 3H), 3.83 (s, 3H), 3.72 (s, 3H), 3.83 (m, 2H), 2.45 (t, J=6.2 Hz, 2H), 1.48 (s, 9H), 1.27 (s, 9H).

### EXAMPLE 3

Preparation of 4-[({4-[({4-[(tert-butoxycarbonyl)amino]-1-methyl-1H-pyrrol-2-yl}carbonyl)amino]-1-methyl-1H-pyrrol-2-yl}carbonyl)amino]-1-methyl-1H-pyrrole-2-carboxylic Acid

To a solution of the compound prepared according to example 2 (3.3 g) in THF (15 ml), a 1N solution of LiOH (3 ml) was added. The solution was stirred at room temperature for 24 hours, the organic solvent was then allowed under vacuum and the residue, treated with water (200 ml), was then extracted with ethyl acetate (2×100 ml). The aqueous phase was acidified with 10% acetic acid, the suspension was filtered under vacuum and the resultant white solid washed with water (20 ml). After desiccation under vacuum at 40° C., the title compound (1.2 g; y=50%) was obtained as an ivory powder.

FAB-MS: m/z 485(100, [M+H]⁺); 491(70), 385(30)

PMR (DMSO-d₆) δ: 9.83 (s, 2H), 9.06 (s, 1H), 7.49 (d, J=1.8 Hz, 1H), 7.20(d, J=1.8 Hz, 1H), 7.06 (d, J=1.8 Hz, 1H), 6.99 (d, J=1.8 Hz, 1H), 6.85 (d, J=1.8 Hz, 1H), 6.65 (d, J=1.8 Hz, 1H), 3.82 (s, 3H), 3.81 (s, 3H), 3.79 (s, 3H), 1.44 (s, 9H).

### EXAMPLE 4

Preparation of tert-butyl 5-{[(5-{[(5-{[(2-{[amino(imino) methyl]amino}ethyl)amino]carbonyl}-1-methyl-1H-pyrrol-3-yl) amino] carbonyl}-1-methyl-1H-pyrrol-3-yl)amino]carbonyl}-1-methyl-1H-pyrrol-3-yl-carbamate hydrochloride

To a solution of the compound prepared according to example 3 (2 g) in dry DMF (50 ml), TEA (0.6 ml), TBTU (1.32 g) and N-(2-aminoethyl)guanidine (0.80 g) were added and the solution was stirred at room temperature for 24 hours. The solvent was allowed under vacuum, the residue treated with water and the suspension filtered in vacuum yielding the title compound (1.5 g; y=60%) as a gummy solid.

FAB-MS: m/z 569(100, [M+H]⁺), 469(25)

PMR (DMSO-d₆) δ: 9.88 (s, 1H), 9.83 (s, 1H), 9.06 (s, 1H), 8.09 (t, J=5.5 Hz, 1H), 7.20 (d, 7=1.8 Hz, 1H), 7.16 (d, J=1.8 Hz, 1H), 7.05 (d, J=1.8 Hz, 1H), 6.96 (d, J=1.8 Hz, 1H), 6.88 (d, J=1.8 Hz, 1H), 6.83 (d, J=11.8 Hz, 1H), 3.83 (s, 3H), 3.81 (s, 3H), 3.80 (s, 3H), 3.40-3.20 (m, 4H), 1.45 (s, 9H).

### EXAMPLE 5

Preparation of 4-amino-N-(5-{[(5-{[(2-{[amino(imino) methyl]amino}ethyl)amino]carbonyl}-1-methyl-1H-pyrrol-3-yl)amino]carbonyl}-1-methyl-1H-pyrrol-3-yl)-1-methyl-1H-pyrrole-2-carboxamide dihydrochloride [compound of formula (I)]

The compound prepared according to example 4 (1.5 g) was dissolved in a HCl/EtOH solution (20 ml) and stirred at room temperature for 12 hours. The solvent was evaporated under vacuum yielding the title compound (1.1 g, y=90%) as a brown powder.

FAB-MS: m/z 469, (15, [M+H]⁺)

PMR (DMSO-d₆) δ: 10.38-10.11 (bs, 4H), 9.98 (s, 1H), 8.28 (bs, 1H), 8.19 (d, J=1.8 Hz, 1H), 7.73, (bs, 1H), 7.63 (d, J=1.8 Hz, 1H), 7.60-7.00 (bs, 4H), 7.28 (d, J=1.8 Hz, 1H), 7.20 (d, J=1.8 Hz, 1H), 7.1 (d, J=1.8 Hz, 1H), 6.92 (d, J=1.8 Hz, 1H), 3.93 (s, 3H), 3.90 (s, 3H), 3.82 (s, 3H), 3.28 (m, 4H).

### EXAMPLE 6

Preparation of N-(5-{[(5-{[(5-{[(2-{[amino(imino)methyl]amino}ethyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide [compound PNU 166196]

A solution of 500 mg of 1-methyl-4-(α-bromoacrylamido) pyrrole-2-carboxyl chloride, prepared as reported in WO 98/04524, in 30 ml of benzene, was added to a solution of the compound prepared according to example 5 (500 mg) and of 164 mg of NaHCO₃ in 10 ml of H₂O. The solution was vigorously stirred for 8 hours at room temperature and then evaporated in vacuum. The crude residue was purified by flash chromatography (dichloromethane/methanol=8/2) to yield 440 mg of the title compound, as a yellow solid.

FAB-MS: m/z 723, (32, (M+H]⁺)

PMR (DMSO-d₆) δ: 10.30 (s, 1H), 9.95 (s, 1H), 9.92 (s, 1H), 9.90 (s, 1H), 8.10 (t, J=5.9 Hz, 1H), 7.56 (t, J=5.9, 1H), 7.2 (bs, 4H), 6.9-7.3 (m, 8H), 6.68 (d, J=2.9 Hz, 1H), 6.21 (d, J=2.9 Hz, 1H), 3.85 (s, 3H), 3.84 (s, 3H), 3.83 (s, 3H), 3.80 (s, 3H), 3.30 (bs, 4H).

1. A process for preparing a poly-pyrroleamido derivative of formula (I) 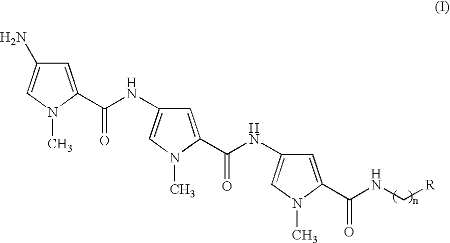
wherein n is an integer from 1 to 4 and R is selected from 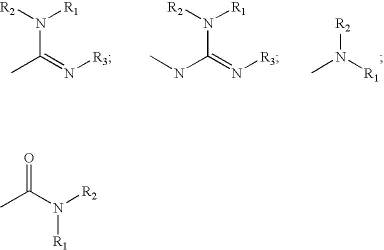
wherein R₁, R₂ and R₃, which are the same or different, are independently selected from hydrogen, methyl and ethyl; which process comprises:
a) reacting distamycin A with succinic anhydride in the presence of a base so as to obtain a compound of formula 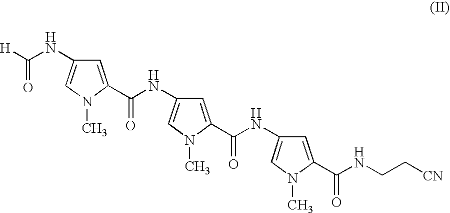
b) reacting the compound of formula (II) with tert-butyl-dicarbonate, in the presence of dimethylaminopyridine (DMAP), so as to obtain a compound of formula 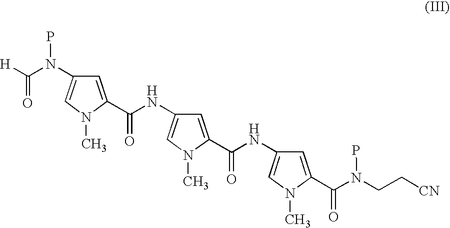
wherein P is tert-butoxycarbonyl;
c) hydrolysing under basic conditions the compound of formula (III) so as to obtain the derivative of formula 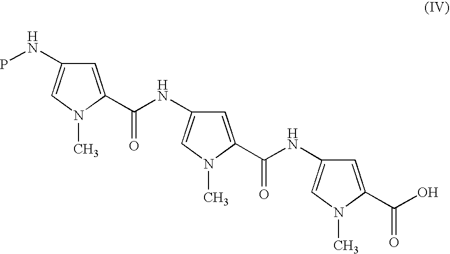
wherein P is as defined above;
d) reacting, in the presence of a suitable coupling agent, the compound of formula (IV) with a compound of formula 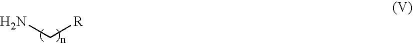
wherein n and R are as defined above; and
e) deprotecting the resulting compound so as to obtain the free amino derivative of formula (I).

2. A process according to claim 1, wherein R is a group of formula 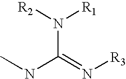
wherein R₁, R₂ and R₃, which are the same or different, are independently selected from hydrogen, methyl and ethyl.

3. A process according to claim 2 wherein each of R₁, R₂ and R₃ is hydrogen.

4. A process for preparing a distamycin derivative of formula 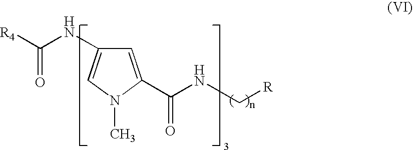
wherein
n is an integer from 1 to 4;
R is selected from 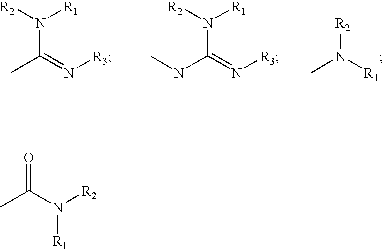
wherein R₁, R₂ and R₃, which are the same or different, are independently selected from hydrogen, methyl and ethyl; R₄ is selected from: 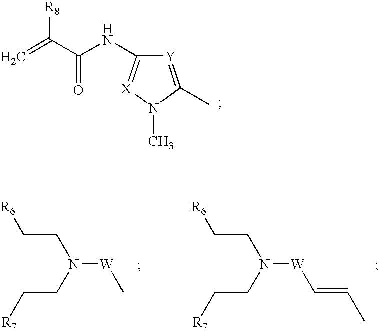
wherein
R₅ and R₆ are chlorine or bromine;
R₇ is hydrogen, chlorine or bromine;
X and Y, which are the same or different, are selected from nitrogen and a CH group;
W is phenylene or a benzocondensed 5 or 6 membered heterocycle containing 1 or 2 heteroatoms selected from N, O and S, both of which are unsubstituted or are further substituted by one or more lower alkyl groups;
or a pharmaceutically acceptable salt thereof;
which process comprises:
a) reacting distamycin A with succinic anhydride in the presence of a base so as to obtain a compound of formula 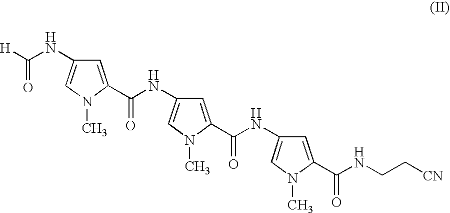
b) reacting the compound of formula (II) with tert-butyl-dicarbonate, in the presence of dimethylaminopyridine (DMAP), so as to obtain a compound of formula 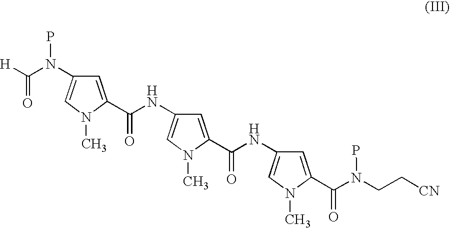
wherein P is tert-butoxycarbonyl;
c) hydrolysing under basic conditions the compound of formula (III) so as to obtain the derivative of formula 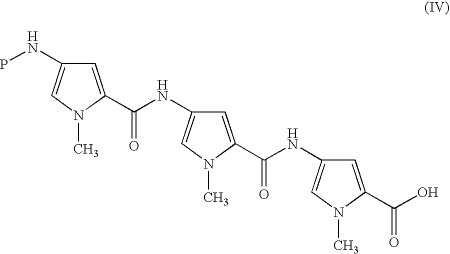
wherein P is as defined above;
d) reacting, in the presence of a suitable coupling agent, the compound of formula (IV) with a compound of formula 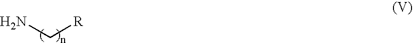
wherein n and R are as defined above;
e) deprotecting the resulting compound so as to obtain the free amino derivative of formula (I) 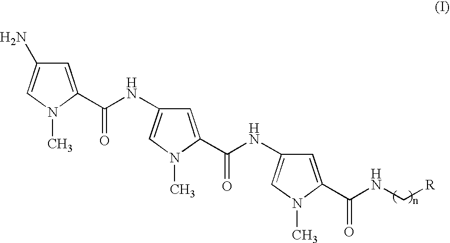
wherein n and R are as defined above; and
f) acylating the compound of formula (I) with a carboxylic acid derivative of formula
R₄—COZ (VII)
wherein R₄ is as defined above and Z is hydroxy or a suitable leaving group, so as to obtain a compound of formula (VI) and, if desired, converting a said compound into a pharmaceutically acceptable salt thereof.

5. A process according to claim 4, wherein R represents a group of formula 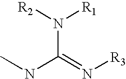
wherein R₁, R₂ and R₃, which are the same or different, are selected from hydrogen, methyl and ethyl; R₄ is an α-bromo- or α-chloro-acryloyl moiety of formula 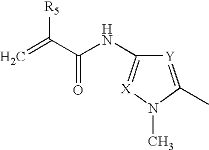
wherein R₅ is chlorine or bromine; X and Y, which are the same or different, are selected from nitrogen and a CH group.

6. A process, according to claim 4, wherein the compound prepared is N-(5-{((5-{[(5-{[(2-{(amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl) amino]-1-methyl-1H-pyrrole-2-carboxamide, optionally in the form of a pharmaceutically acceptable salt.

7. A process according to any one of the preceding claims wherein, in step b), from 2 to 4 equivalents of di-tert-butyldicarbonate and from 2 to 4 equivalents of dimethylaminopyridine (DMAP), are used.

8. A process according to any one of claims from 1 to 6 wherein, in step d), the reaction between the compound of formula (IV) and the compound of formula (V) is carried out in the presence of a suitable coupling agent selected from N,N′-dicyclohexylcarbodiimide (DCC), 1-(3-dimethylaminopropyl)-3-ethylcarbodiimide hydrochloride (EDCI), benzotriazol-1-yloxytris(dimethylamino)phosphonium hexafluorophosphate (BOP) and O-(1H-benzotriazol-1-yl)-N,N,N′,N′-tetramethyluronium tetrafluoroborate (TBTU).

9. A process according to claim 8 wherein the coupling agent is O-(1H-benzotriazol-1-yl)-N,N,N′,N′-tetramethyluronium tetrafluoroborate (TBTU).

10. A process according to any one of claims 1 to 6 wherein, in step e), the removal of the amino protecting tert-butoxycarbonyl group is carried out under acidic conditions, in the presence of hydrochloric or trifluoroacetic acid.

11. A process according to claim 6 wherein, in step f), the compound of formula (I) is reacted with a compound of formula (VII) wherein Z is hydroxy, bromine or chlorine.
